(19) United States
(12) Patent Application Publication
(10) Pub. No.: US 2006/0219472 A1
(43) Pub. Date: Oct. 5, 2006

(54) Stetoscope holder

(76) Inventor: Stephen G. Vance, Dillon, SC (US)

Correspondence Address: LITMAN LAW OFFICES, LTD, PO BOX 15035, CRYSTAL CITY STATION, ARLINGTON, VA, 22215, US

(21) Appl. No.: 11/369,743

(22) Filed: Mar. 8, 2006

Related U.S. Application Data
(60) Provisional application No. 60/666,160, filed on Mar. 29, 2005.

(51) Int. Cl. A61B 7/02; A45C 1/04

(52) U.S. Cl. 181/131; 224/670

(57) ABSTRACT

The stethoscope holder is a clip that grips a belt or cincture of an article of clothing and holds a stethoscope. The holder has a first arm biased against a second arm by a spring, such as a flat or leaf spring formed in a V-shape. A hinge or pivot pin holds the two arms together in a pivoting manner. The first arm has a front surface and a rear surface with a finger grip extending from the front surface at an angle. A gripping surface is formed on the post, the front surface, and the rear surface of the first arm. The second arm may be J-shaped. The second arm has a front surface and a rear surface. A gripping surface is formed on the front surface of the rear arm. The gripping surfaces may be formed by ribs, ridges, or flutes.

### CROSS-REFERENCE TO RELATED APPLICATION

This application claims the benefit of U.S. Provisional Patent Application Ser. No. 60/666,160, filed Mar. 29, 2005.

### BACKGROUND OF THE INVENTION

1. Field of the Invention

The present invention relates to instrument holders, and more specifically to a stethoscope holder that can be clipped to a belt or other waist cincture.

2. Description of the Related Art

Many health care professionals have frequently used medical instruments that are carried upon their person for ready or instant retrieval. For example, stethoscopes are generally wound up and placed in pockets of hospital jackets or suspended from a user's neck until the stethoscope is needed. When the stethoscope is wound and placed in a pocket, the parts of the stethoscope may separate or the tubing may become damaged; the user may further find it difficult to grasp the stethoscope and untangle the tubing from other items carried in the pocket. In addition, some garments may not have a pocket of sufficient size or location to receive the stethoscope.

As a result, the stethoscope is frequently placed around the user's neck for easy access. This, however, can place unnecessary weight on the nape of the neck, or become a hazard to the user when the stethoscope is accidentally detached and falls from the neck. The earpieces may also rub against the neck, causing annoyance and possible discomfort. Thus, a stethoscope holder solving the aforementioned problems is desired.

### SUMMARY OF THE INVENTION

The stethoscope holder is a clip that grips a belt or waistband of an article of clothing and holds a stethoscope. The holder permits the user to retain the stethoscope without having to wear the stethoscope around the user's neck. The holder has a first arm biased against a second arm by a spring, such as a flat or leaf spring formed in a V-shape to apply torsion to the opposing arms. A pin holds the two arms together in a pivoting manner.

The first arm has a front surface and a rear surface with a finger grip, such as a post, extending from the front surface at an angle. A gripping surface is formed on the post, the front surface and the rear surface of the first arm. The second arm may be J-shaped. The second arm has a front surface and a rear surface. A gripping surface is formed on the front surface of the rear arm. The gripping surface may be formed by any means that provides for gripping, such as axially extending ribs, ridges, or flutes.

These and other features of the present invention will become readily apparent upon further review of the following specification and drawings.

### BRIEF DESCRIPTION OF THE DRAWINGS

FIG. 1 is an environmental, perspective view of a stethoscope holder according to the present invention showing the holder attached to a belt.

FIG. 2 is a perspective view of the stethoscope holder according to the present invention.

FIG. 3 is an exploded perspective view of the stethoscope holder according to the present invention.

FIG. 4 is a rear perspective view of the first arm of the stethoscope holder according to the present invention.

FIG. 5 is an environmental, perspective view of a stethoscope holder according to the present invention showing the holder attached to a waistband.

FIG. 6 is a rear view of the stethoscope holder according to the present invention.

Similar reference characters denote corresponding features consistently throughout the attached drawings.

### DETAILED DESCRIPTION OF THE PREFERRED EMBODIMENT

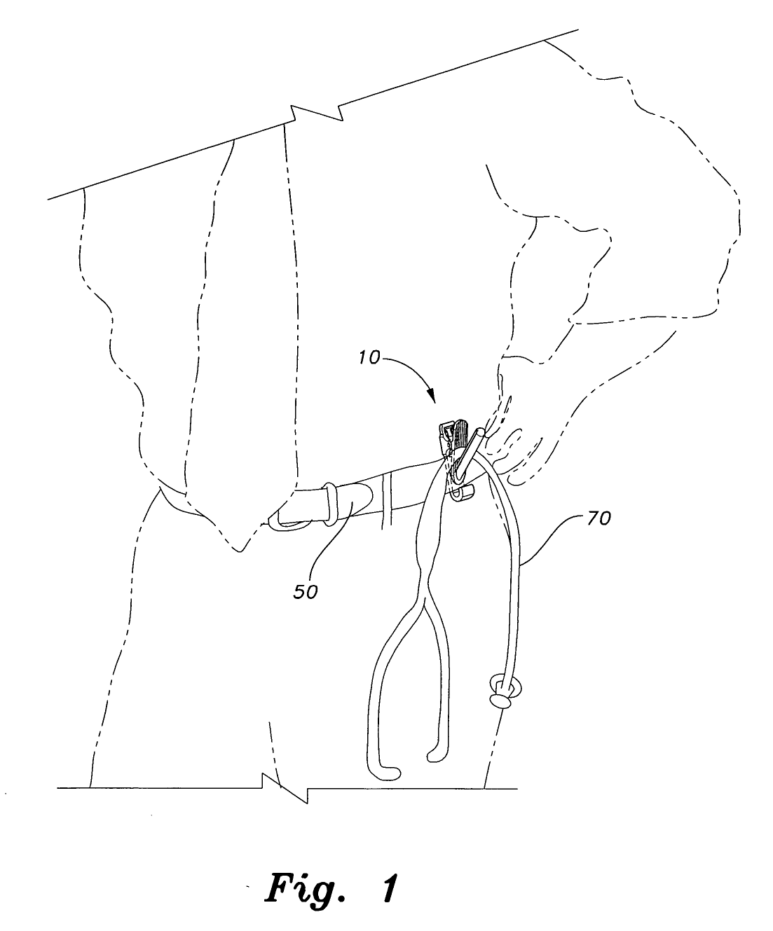

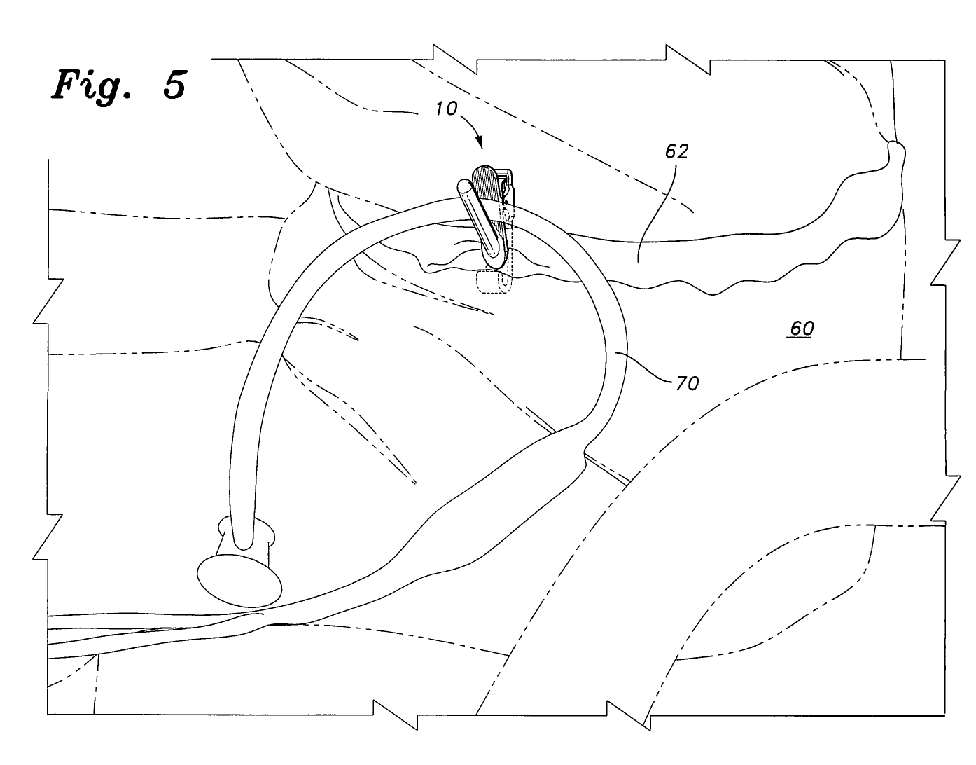

The present invention is a stethoscope holder designated generally as 10 in the Figures. As shown in FIGS. 1 and 5, the holder 10 clips onto a belt 50 or an edge 62 or other cincture of an article of clothing 60, such as a waistband on scrub pants or a skirt. The holder 10 retains a stethoscope 70 when not in use and permits a user to carry the stethoscope 70 on his or her person without having to wear the stethoscope 70 around the user's neck.

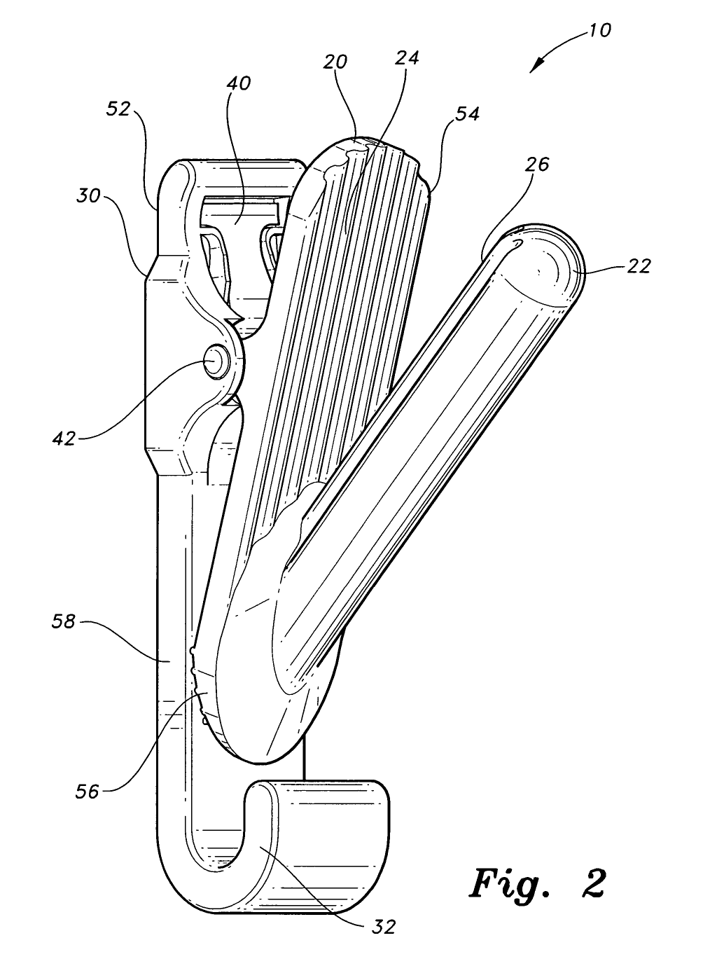

Referring to FIGS. 2-4, the holder 10 has a first arm 20 biased against a second arm 30 by a spring or other suitable elastic element 40, such as a flat or leaf spring formed in a V-shape to apply torsion to the opposing arms 20 and 30. A hinge or pivot pin 42 holds the two arms 20, 30 together and allows the arms 20, 30 to pivot from a normally closed position to an open position in order to attach the holder 10 to a belt or waistband.

The first arm 20 has an upper end 54, a lower end 56, a front surface and a rear surface, with a finger grip, such as a post 22, extending at an angle from the front surface. A gripping surface 24 is formed on the front surface of the first arm 20. The gripping surface 24 may be formed by any means that provides for gripping a stethoscope, e.g., axially extending ribs, ridges, or flutes that are adapted to bear against and press into the flexible tubing of the stethoscope to frictionally retain the stethoscope between the first arm and the post 22.

The finger grip may have any shape that permits its use to apply torque to pivot and separate the first arm 20 and the second arm 30 to attach the holder 10 to a belt, but which also forms a notch with the first arm into which the stethoscope is inserted and wedged. In the drawings, the finger grip is shown as post 22, which is a cylindrical rod designed to carry the weight of the stethoscope 70. A gripping surface 26, e.g., axially extending ribs, ridges, or flutes, are formed along at least a portion of the post 22. In particular, a gripping surface 26 is formed at least on the side of the post 22 that faces the front surface of the first arm 20. Gripping surfaces 24 and 26, therefore, face each other in order to retain the stethoscope therebetween.

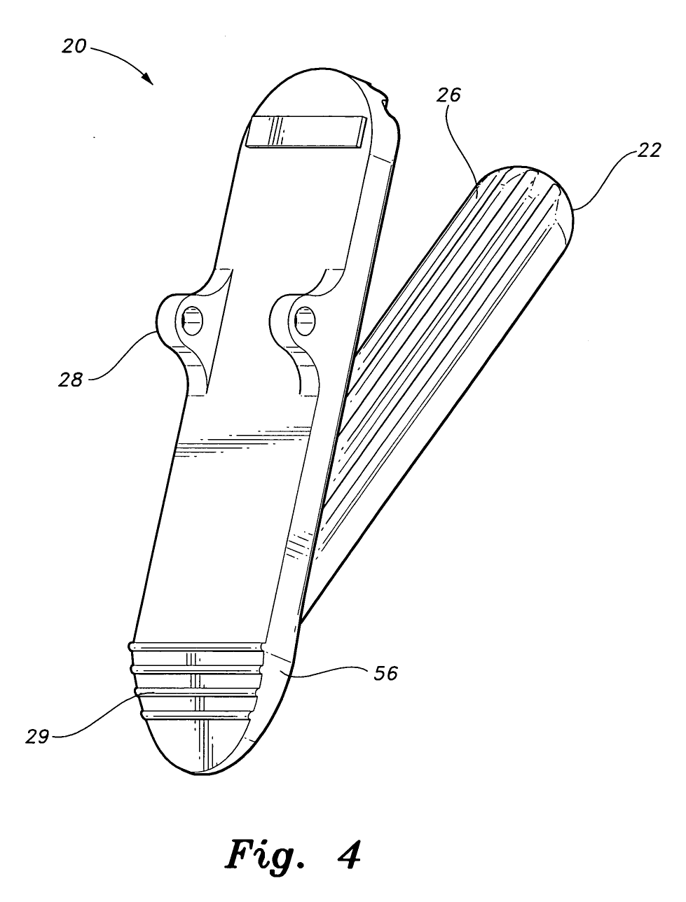

FIG. 4 shows the rear surface of the first arm 20. A gripping surface 29, e.g., transverse ribs, is formed toward the lower end 56 of the first arm 20. A pair of lugs 28 extend from the rear surface of the first arm 20. The rear surface of the first arm may also have a recess formed therein or a stop projecting therefrom for retaining a portion of the spring 36.

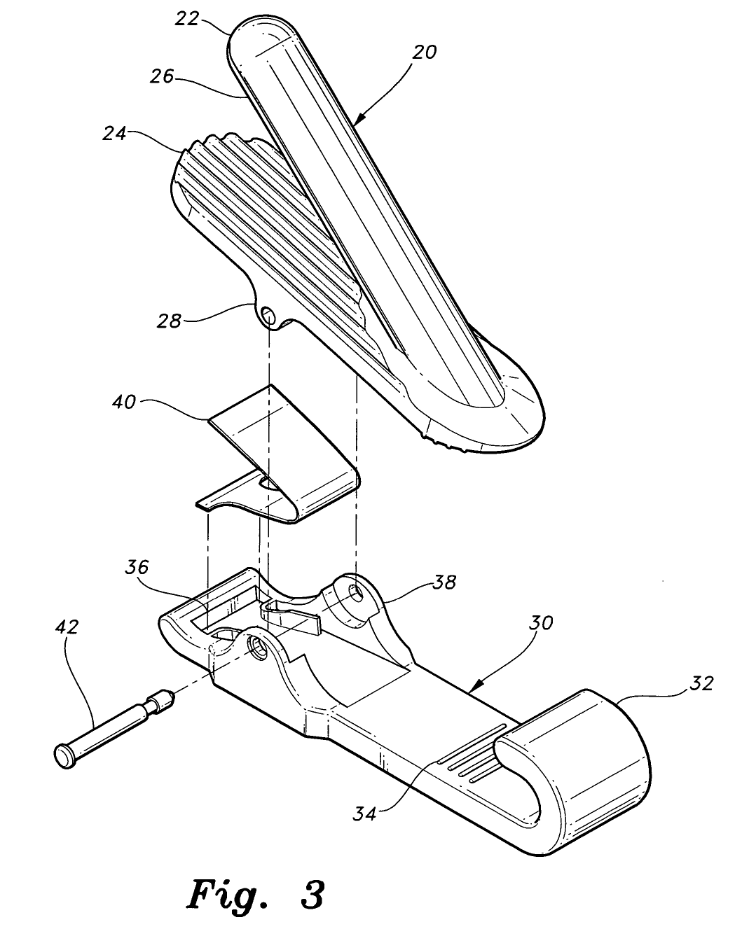

Referring back to FIGS. 2 and 3, the second arm 30 includes an elongated plate and may be J-shaped, having a planar end 52, hooked end 32 and a planar intermediate portion 58 disposed between the top end 52 and the hooked end 32. The second arm 30 also has a front surface and a rear surface. A gripping surface 34, e.g., transverse ridges or ribs, is formed on the front surface of the rear arm 30.

The front surface of the second arm 30 has a cavity or recess 36 for receiving a flat or leaf spring 36. The front surface of the second arm 30 also has lugs 38 that correspond to lugs 28 of the first arm 20. To form the holder 10, first the spring 40 is set in the cavity 36 of the second arm 30. Next, the lugs 28 of the first arm 20 are aligned with the lugs 38 of the second arm 30 to sandwich the spring 40 between the arms 20 and 30 and a hinge or pivot pin 42 is inserted through openings in both lugs 28, 38 to pivotally join the arms 20, 30 in a closed position, the pin 42 extending between the legs of the V-shaped spring 36 adjacent the vertex. It will be understood that although a V-shaped flat or leaf spring 36 is shown in the drawings, any resilient means, e.g., a torsion spring disposed about hinge pin 42, a helical spring between arms 20 and 30, etc., for biasing the upper ends of arms 20 and 30 apart and pivoting gripping surfaces 29 and 34 together may be used in lieu of flat spring 36.

The holder 10 frees the user from the necessity of hanging the stethoscope 70 around the user's neck for quick access. The holder 10 may be disposed either on the belt 50 or other cincture of articles of clothing 60, such as on the waistband 62 on pants 60 or a skirt, or possibly on a collar of a scrub top, etc. When the holder 10 is disposed on the belt 50, the belt 50 is sandwiched by the two arms 20, 30 with the hooked end 32 cupping a bottom edge of the belt 50. Depending upon the tension exerted by spring 36, the holder 10 may slide along the length of the belt 50 or grip the belt 50 firmly to preclude sliding. When the holder 10 is disposed on the waistband 62 of the article of clothing 60, the holder 10 clamps the waistband 62 between the two arms 20, 30 with the hooked end 32 being hidden. Regardless of whether the holder 10 is attached to the belt 50 or the article of clothing 60, the gripping surfaces 29 and 34 on the first and second arms 20, 30, respectively, provide friction for keeping the holder 10 in position.

The post 22 juts out from the front surface of the first arm 20 and is designed to bear the weight of the stethoscope 70. Preferably, the stethoscope 70 is held at its mid-section so that half of the weight of the stethoscope 70 hangs over each side of the post 22. The gripping surfaces 24 and 26 disposed on the first arm 20 and the post 22 provide impinge upon the flexible tubing of the stethoscope 70 to preclude the stethoscope 70 from sliding off the holder 10.

The holder 10 may be made in any size and the post 22 may be made to have any length and width. A longer post 22 provides the user with a place to accommodate more than one stethoscope 70. The holder 10 has been shown as a device for holding stethoscopes 70; however the holder 10 may be used for other purposes. For instance, it is possible to use the holder 10 as a wire holder by a handy person or an electrician as a way to keep wires close to the user's body, or for carrying other elongated flexible tubes, conduits, and the like. Likewise, the holder may be useful in holding other articles, such as a key ring of a key fob, as well as other articles that can be carries about the waist.

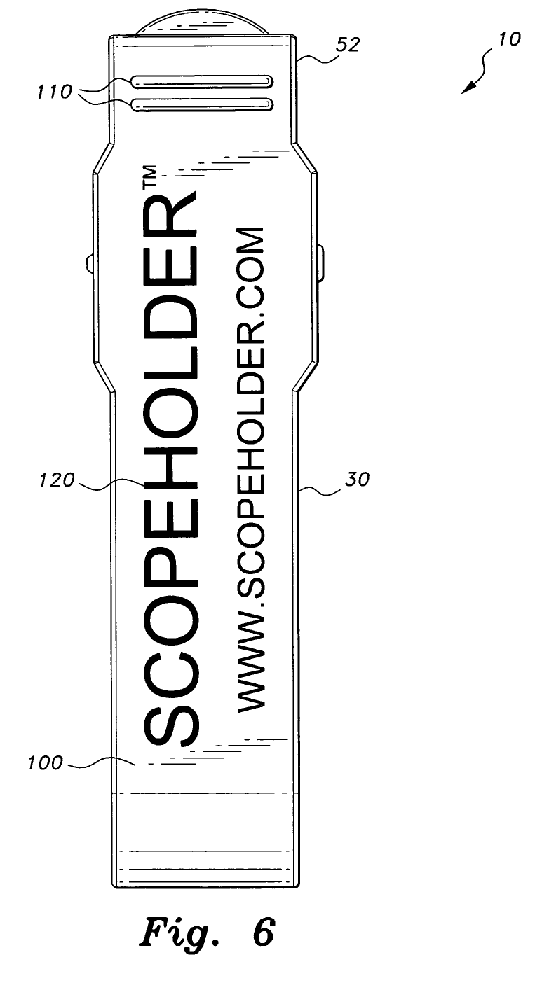

Additionally, as shown in the rear view of FIG. 6, the rear face 100 of second arm 30 may have indicia 120 imprinted thereon by any suitable method. Indicia 120 may be a trademark, trade name or any suitable indicia, depending upon the needs and desires of the user. Further, the upper end 52 of second arm 30 may include ribs 110, similar to gripping surface 34, positioned on rear face 100, as shown. Though shown as being two horizontally positioned ribs, it should be understood that ribs 110 may have any suitable number and orientation, depending upon the needs and desires of the user.

It is to be understood that the present invention is not limited to the embodiments described above, but encompasses any and all embodiments within the scope of the following claims.

I claim:

1. A holder, comprising:
a first arm having a finger grip extending therefrom at an angle forming a notch;
a second arm pivotally joined to the first arm;
means for biasing a lower end of the first arm against a lower end of the second arm; and
gripping means formed on opposing surfaces of the first arm and the finger grip for retaining an article wedged in the notch.

2. The holder according to claim 1, wherein said second arm has a substantially J-shaped cross-sectional contour.

3. The holder according to claim 1, wherein said means for biasing is a leaf spring.

4. The holder according to claim 1, wherein said gripping means comprise a plurality of grooves formed in said opposing surfaces.

5. The holder according to claim 4, wherein each of said plurality of grooves extends in a substantially longitudinal direction.

6. The holder according to claim 1, further comprising a plurality of first ribs formed on a rear surface of said first arm adjacent said lower end thereof for frictionally engaging an article of clothing.

7. The holder according to claim 6, further comprising a plurality of second ribs formed on a front surface of said second arm adjacent said lower end thereof for frictionally engaging said article of clothing.

8. The holder according to claim 7, wherein said first and second ribs extend in a substantially lateral direction.

9. The holder according to claim 1, wherein a rear surface of said second arm is adapted for having indicia imprinted thereon.

10. A stethoscope holder, comprising:
a first arm having a finger grip extending therefrom at an angle forming a notch;
a second arm pivotally joined to the first arm;
means for biasing a lower end of the first arm against a lower end of the second arm; and
gripping means formed on opposing surfaces of the first arm and the finger grip for retaining a stethoscope wedged in the notch.

11. The stethoscope holder according to claim 10, wherein said second arm has a substantially J-shaped cross-sectional contour.

12. The stethoscope holder according to claim 10, wherein said means for biasing is a leaf spring.

13. The stethoscope holder according to claim 10, wherein said gripping means comprise a plurality of grooves formed in said opposing surfaces.

14. The stethoscope holder according to claim 13, wherein each of said plurality of grooves extends in a substantially longitudinal direction.

15. The stethoscope holder according to claim 10, further comprising a plurality of first ribs formed on a rear surface of said first arm adjacent said lower end thereof for frictionally engaging an article of clothing.

16. The stethoscope holder according to claim 15, further comprising a plurality of second ribs formed on a front surface of said second arm adjacent said lower end thereof for frictionally engaging said article of clothing.

17. The stethoscope holder according to claim 16, wherein said first and second ribs extend in a substantially lateral direction.

18. The stethoscope holder according to claim 10, wherein a rear surface of said second arm is adapted for having indicia imprinted thereon.

19. The stethoscope holder according to claim 10, further comprising a plurality of ribs formed on a rear surface of said second arm for frictionally engaging an article of clothing.

20. The stethoscope holder according to claim 19, wherein said plurality of ribs are positioned adjacent an upper end of said second arm.
